(19) United States
(12) Patent Application Publication
(10) Pub. No.: US 2005/0251109 A1
(43) Pub. Date: Nov. 10, 2005

(54) Device for locking/unlocking rotation in the organism

(76) Inventor: Arnaud Soubeiran, Paris (FR)

Correspondence Address: SUGHRUE MION, PLLC, 2100 PENNSYLVANIA AVENUE, N.W., SUITE 800, WASHINGTON, DC, 20037, US

(21) Appl. No.: 10/525,716

(22) PCT Filed: Aug. 29, 2003
(86) PCT No.: PCT/FR03/02615

§ 371 Date: Feb. 25, 2005

(30) Foreign Application Priority Data
Aug. 30, 2002 (FR) 02/10746

(51) Int. Cl. A61B 17/00

(52) U.S. Cl. 606/1

(57) ABSTRACT

The invention concerns a device for non-invasive locking/unlocking rotation between first (1) and second (2) components implanted in the organism comprising first locking surfaces (11) integral with the first component (1), second locking surfaces (21) integral with the second component (2), at least one locking component (3) adapted to co-operate in locked state with both said first (11) and second (21) locking surfaces and in a released state with not more than one of said surfaces (11, 21) and to shift from one state to the other under the effect of the gravitational field or of a magnetic field in at least one specific mutual angular position of the first (1) and second (2) components. Means such as a flexible conduit (411) ensure sealing conditions of the volume wherein the locking component (3) moves. The device enables the control of the elongation obtained by a rotary centro-medullar nail or a growth prosthesis.

The present invention relates to the non-invasive control of implantable systems with adjustable geometry inside the organism.

Several systems with adjustable geometry inside the organism comprise a part the rotation of which in one direction and in the other causes the geometry of the said system to be adjusted. This is the case, for example, with bone lengthening nails, such as those described in documents U.S. Pat. No. 5,074,882 or U.S. Pat. No. 5,505,733, which transform rotation in one direction and in the other of one part relative to another into a lengthening, or even constrained knee prostheses, in which the rotation of the tibial part or the femoral part in one direction and in the other relative to an axis allows the geometry to be adjusted.

However, there is no simple non-invasive means to block these systems in a given position and then to unblock them to enable them to continue their adjustment when this is desired.

The device according to the invention thus proposes a simple and effective solution to block and unblock in a non-invasive manner the rotation in one direction and in the other between two first and second parts implanted in the organism, since it comprises:
first blocking surfaces bound to the said first part,
second blocking surfaces bound to the said second part,
at least one blocking part:
   which comprises third blocking surfaces, able to cooperate with the said first blocking surfaces which are bound to the said first part and fourth blocking surfaces, able to cooperate with the said second blocking surfaces bound to the said second part, and
   capable, when both the first and second said parts are at a particular angular position, of passing from a first free state in which both the first and second said parts are free to rotate relative to one another, with at least one of the said third and fourth blocking surfaces of the said blocking part being free from the blocking surfaces of that of the said first and second parts with which they can cooperate, to a second blocked state in which the said first and second parts are prevented from rotating relative to one another by means of the said first and third blocking surfaces firstly, and of the said second and fourth blocking surfaces secondly, and vice-versa to pass from the said second blocked state to the said first free state.
means defining between the said first and second parts a cavity sealed against the surrounding materials which includes at least the said first and second blocking surfaces and the volume swept by the said blocking part when it moves between the said first free state and the said second blocked state, and vice-versa,
means to impart rotation in one direction and in the other from outside the organism to the two said first and second parts relative to one another,

The blocking part may also comprise:
an element over which a magnetic field is able to exert a force,
means to hold the said blocking part in at least one of the said first free state and second blocked state.

The said means defining between the said first and second parts a cavity sealed against the surrounding materials can be of any type and notably:
at least one flexible conduit bound at one of its ends to the said first part and at the other of its ends to the said second part. In this latter case, the rotational amplitude allowed between the said first and second parts must be limited in order not to exceed the elastic deformation or fatigue limit during the planned service of the said flexible conduit.
at least one joint, for example an O-ring joint, placed directly between the two said first and second parts if they have faces perpendicular to their relative axes of rotation placed one against the other,
a third part which surrounds the two said first and second parts and joints, for example O-ring joints, placed between respectively the said third part and the said first part, and the said third part and the said second part,

The said means to impart rotation in one direction and in the other from outside the organism to the two said first and second parts relative to one another can, notably, be constituted by means to link respectively the said first and second part, each to a different part of the organism, such as a bone or a bone segment, which are able to be manipulated separately from outside the organism, something which will be particularly advantageous in an bone-lengthening nail or expandable prosthesis application, for example. They can also be constituted by one or more parts placed under the skin and able to be manipulated through it in one direction at least, wherein the other direction is able to be provided by elastic means which have been compressed during the manipulation in the first direction, for example.

In the remainder of the present description, for greater clarity, “the free state” will be put for “the said first free state” and “the blocked state” for “the said second blocked state”.

The said blocking surfaces are of the male-female type, but can take very varied shapes, dimensions and positions (cylinder in perforation, cross in cruciform cavity, parallelepiped lamella in parallelepiped cavity, etc.). They are oriented so as to allow the movement of transition from the free state to the blocked state and vice-versa, and a sufficient play, depending on the characteristics of the materials used and the dimensions of the device, is left between two groups of blocking surfaces able to cooperate with one another to facilitate the said movements. Those skilled in the art will understand without difficulty the surfaces suited to a given problem as a function notably of the efforts which they must resist, the encumbrance of the device and of the available manufacturing techniques.

The movement to change the blocking part from the free state to the blocked state or vice-versa when the said first and second parts are in a particular angular position may be a translation, a rotation or any movement guided by surfaces such as those also sealing the volume, whether or not swept.

The said transition of the said blocking part from the free state to the blocked state or vice-versa occurs when the force exerted on the said blocking part by the field of gravity, or a magnetic field if the said blocking part comprises an element which is sensitive to it, is suitably oriented and the said first and second parts have been positioned so as to reduce sufficiently the efforts being exerted by means of the said first and second blocking surfaces on the said blocking part, and which are opposed to the said transition.

There may be several discrete particular angular positions relative to the said first and second parts, each allowing the transition of the said blocking part from the free state to the blocked state, or vice-versa. This is the case in particular if at least one of the blocking surfaces has a symmetry around the axis of rotation of the said first and second parts.

The invention, its operation and its applications will be better understood, and others of its characteristics and advantages will be revealed, during the following description made on sight of the illustrations annexed for illustrative purposes, but by no means on a limitative basis, in which:

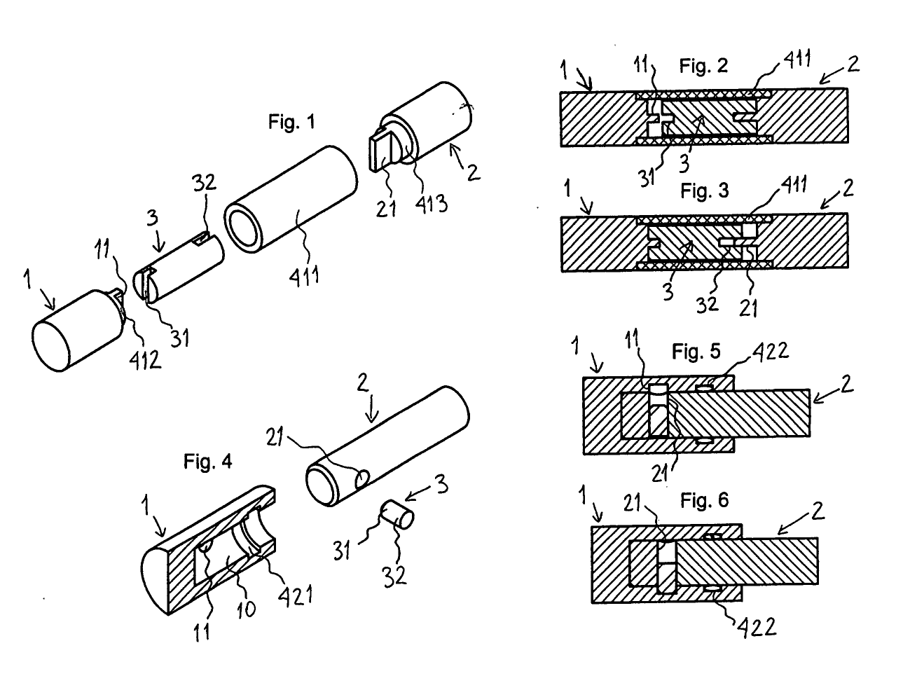

FIGS. 1 to 3 represent a preferred embodiment of the device according to the invention.

FIG. 1 is an exploded perspective view of this embodiment.

FIGS. 2 and 3 are cross-section views respectively in the free state and in the blocked state.

FIGS. 4 to 6 represent a second embodiment of the device according to the invention.

FIG. 4 is an exploded perspective and partially cut-away view of this second embodiment.

FIGS. 5 and 6 are cross-section views respectively in the free state and in the blocked state.

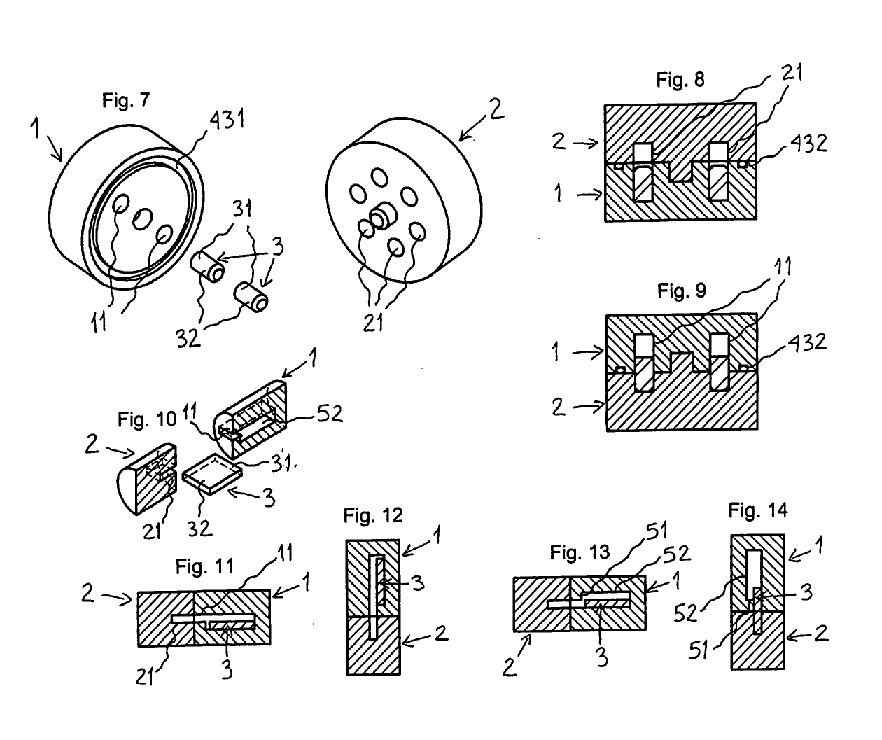

FIGS. 7 to 9 represent a third embodiment of the device according to the invention, in which the said second blocking surfaces allow the transition of the said blocking part from the free state to the blocked state in several particular angular positions.

FIG. 7 is an exploded perspective view of this third embodiment.

FIGS. 8 and 9 are cross-section views in a particular angular position respectively in the free state and in the blocked state.

FIGS. 10 to 14 represent a fourth embodiment of the device according to the invention which comprises means to hold the said blocking part in the free state.

FIG. 10 is an exploded perspective and partially cut-away view of this fourth embodiment.

FIGS. 11 to 14 are cross-section views of it.

FIGS. 11 to 13 represent the said fourth embodiment in the free state in various circumstances.

FIG. 14 represents it in the blocked state.

It is specified that, in these figures, the same references designate the same elements, whatever the figure in which they appear, and whatever the form of representation of these elements. Similarly, if elements are not specifically referenced in one of the figures, the reference can easily be found by referring to another figure.

In all the figures the hidden lines are invisible, except in the case of parts for which they facilitate understanding. In all the figures the dimensions and proportions have been altered when this was able to facilitate understanding.

In all the figures, the magnetic or gravitational force which can be exerted on the said blocking part is presumed to be directed from the top to the bottom of the figure.

The applicant is also keen to specify that the figures represent several embodiments of the object according to the invention, but that other embodiments exist satisfying the definition of this invention.

He also specifies that when, according to the definition of the invention, the object of the invention comprises “at least one” element with a given function, the described embodiment may comprise several of these elements.

He also specifies that if the embodiments of the object according to the invention as illustrated comprise several elements with identical functions and if, in the description, it is not specified that the object according to the invention must in all cases comprise a particular number of these elements, the object of the invention may be defined as comprising “at least one” of these elements.

He specifies, finally, that the means to mount the said first and second parts such that they may rotate relative to each other, which may be very varied, and are easily conceived by those skilled in the art according to the system in which the said first and second parts are incorporated, depend on the said system, and their nature is not a factor in the operation of the said device according to the invention. They have not therefore been represented. Similarly, the said first and second parts may have very varied shapes, and only their parts involved in the said device according to the invention have been represented.

The device according to the invention to block and unblock in a non-invasive manner the rotation between two first 1 and second 2 parts implanted in the organism, in all its embodiments and notably those represented in FIGS. 1 to 14, comprises:
first blocking surfaces 11 bound to the said first part 1,
second blocking surfaces 21 bound to the said second part 2,
at least one blocking part 3:
   which comprises third blocking surfaces 31, able to cooperate with the said first blocking surfaces 11 which are bound to the said first part 1 and the fourth blocking surfaces 32, able to cooperate with the said second blocking surfaces 21 bound to the said second part 2, and
   capable, when both the said first 1 and second 2 parts are at a particular angular position, of passing from a first free state in which both the said first 1 and second 2 parts are free to rotate relative to one another, with at least one of the said third 31 and fourth 32 blocking surfaces of the said blocking part being free from the blocking surfaces 11, 21 of that of the said first 1 and second 2 parts with which they can cooperate, to a second blocked state in which the said first 1 and second 2 parts are prevented from rotating relative to one another by means of the said first 11 and third 31 blocking surfaces firstly, and of the said second 21 and fourth 32 blocking surfaces secondly, and vice-versa to pass from the said second blocked state to the said first free state.
means 411, 412, 413 or 421, 422 or 431, 432 defining between the said first 1 and second 2 parts a cavity sealed against the surrounding materials which includes at least the said first 11 and second 21 blocking surfaces and the volume swept by the said blocking part 3 when it moves between the said first free state and the said second blocked state, and vice-versa,
means to impart rotation in one direction and in the other from outside the organism to the said first and second parts relative to one another,

The said means to impart rotation in one direction and in the other from outside the organism to the two said first and second parts relative to one another may be very varied, are a function of the system in which the device according to the invention is incorporated, are in general known or easily definable by those skilled in the art, and are not thus represented.

According to a preferred embodiment of the device according to the invention represented in FIGS. 1 to 3, the said first 1 and second 2 parts each have an end constituted by a cylinder, respectively 412, 413, prolonged by substantially parallelepiped forms the faces of which constitute respectively the first 11 and second 21 blocking surfaces. Each of these ends is symmetrical relative to the axis of rotation of the said first 1 and second 2 parts. They are also turned to face one another and separated by a distance greater than the length according to the axis of rotation of the said first 1 and second 2 parts of the said parallelepiped of the first part 1 which is at most half that according to the same axis of the said parallelepiped of the second part 2, which enables the blocking part 3 to be inserted between the two said ends and operate between them in the manner specified below. The two cylinders 412, 413 have the same diameter.

The blocking part 3 is a cylinder of diameter less than the common one of the cylinders 412, 413 of the ends of the said first 1 and second 2 parts and of length less than the distance between these said two cylinders 412, 413, minus the length according to the axis of rotation of the said first 1 and second 2 parts of the parallelepiped of the said first part 1 and greater than the distance between the two said parallelepipeds respectively of the first part 1 and of the second part 2. The said blocking part 3 also comprises at one end a cavity which is additional to the parallelepiped of the said first part 1, the faces of which constitute the said third blocking surfaces 31 and, at the opposite end, a cavity which is additional to the parallelepiped of the said second part 2, the faces of which constitute the said fourth blocking surfaces 32.

A flexible tube 411, for example made of silicon elastomer, of internal diameter in the absence of constraints substantially equal to that of the cylinders of the said first 1 and second 2 parts, is bound in a sealed manner, for example by gluing after dilatation by an appropriate solvent, at one of its ends on to cylinder 412 of the said first part 1 and at the other on to cylinder 413 of the second part 2, preferably in an angular position of the first 1 and of the second 2 parts which allows the blocking part 3 to change from the free state to the blocked state, or vice-versa, thus constituting the means defining between the said first 1 and second 2 parts a cavity sealed against the surrounding materials. This flexible tube 411 is twisted during the rotations between the first 1 and the second 2 parts.

The entire device is symmetrical relative to the axis of rotation of the said first 1 and second 2 parts.

The blocked state can thus be obtained in two relative positions, at 180° one to the other, of the said first 1 and second 2 parts.

The operation of the device in the gravity field is now described. Operation in a magnetic field if the blocking part comprises an element on which such a field can exert a force is deduced very simply from this and as a consequence will not be explained in greater depth.

The transition from the free state represented in FIG. 2 in which the said first blocking surfaces 11 are completely released from the said third blocking surfaces 31 to the blocked state represented in FIG. 3 in which the third blocking surfaces 31 are overlapped with the first blocking surfaces 11 and simultaneously the fourth blocking surfaces 32 remain overlapped with the second blocking surfaces 21 is accomplished by placing the second part 2 above the first part 1 and by slowly rotating one of the two first 1 or second 2 parts relative to the other in one direction or the other, equally, but with a sufficient amplitude to guarantee that the first 11 and third 31 blocking surfaces will be correctly positioned opposite one another at a given moment such that the blocking part 3 can then be displaced in a translation movement to the blocked state under the effect of gravity.

The transition from the blocked state represented in FIG. 3 to the free state represented in FIG. 2 is accomplished by placing the first part 1 above the second part 2 and by rotating gently one of the first 1 or second 2 parts relative to the other in one direction and then in the other in general, since it is not in principle possible to know the last rotational direction of the system, which is moreover invisible, and because by undertaking the rotation in one direction followed by the other, starting with the one one wishes to have, it is guaranteed that at one time the efforts on the blocking part, which will no longer have any torque to transmit when one of the reversals of direction occurs, are cancelled and that it is then possible to move using a translation movement to the free state under the effect of gravity.

In the second embodiment of the device according to the invention represented in FIGS. 4 to 6, the said first part 1 comprises an attachment hole 10 which receives the end of the said second part 2, which is a cylinder of slightly smaller diameter than that of the said attachment hole 10. The axis of rotation of the said first 1 and second 2 parts is thus that of the said attachment hole. The means defining between the said first 1 and second 2 parts a cavity sealed against the surrounding materials are constituted by a recess 421 close to the emerging end of attachment hole 10 and which receives for example an O-ring joint 422, for example made from silicon elastomer. The blocking part is a cylinder 3 capable of moving between the first blocking surfaces 11 constituted by a non-emerging perforation, of length less than that of the said blocking part 3 and made beyond the said recess 421, for example by spark machining, from the inside of the said attachment hole 10 perpendicular to the axis of the latter and of the second blocking surfaces 21 constituted by a perforation made in the said second part 2 such that there is at least one angular position of the said first 1 and second 2 parts in order that the said first 11 and second 21 blocking surfaces may be aligned. The length of the blocking part 3 is such that it can be fully contained in the said second blocking surfaces 21, which constitutes the free state represented in FIG. 5. The blocked state represented in FIG. 6 is that in which the said blocking part 3 is straddling the said first 11 and second 21 blocking surfaces. It is noted that, as for the preferred embodiment, the blocked state can be obtained in two relative positions, 180° from one another, of the said first 1 and second 2 parts.

The third embodiment of the device according to the invention represented in FIGS. 7 to 9 provides a large number of positions in which the blocked state may be obtained, allowing in particular prostheses to be produced an angle of which may be modified quite finely after their implantation if necessary, or again very simple systems to rotate in one direction or the other a screw implanted in the organism with a precise succession alternatively of rotation and inclination movements of the device according to the invention.

The said first 1 and second 2 parts are in contact through faces perpendicular to their common axis of rotation. The first blocking surfaces 11 are constituted by at least one blind perforation along an axis parallel to that of the rotation, but not aligned with it, and of sufficient depth for a cylindrical blocking part 3 of diameter slightly less than that of the perforation to be able to be fully lodged in it. In FIGS. 7 to 9 two perforations have been represented in which two blocking parts 3 are lodged. The second blocking surfaces 21 are constituted by a number of perforations along axes parallel to the axis of rotation of the said first 1 and second 2 parts, of diameter substantially identical to that of the perforations constituting the said first blocking surfaces 11, but of length less than that of the cylindrical blocking parts 3 and distributed around the said axis of rotation such that there is at least one position of the first part 1 relative to the second part 2 in which each perforation of the first part 1 is opposite a perforation of the second part 2. In FIGS. 7 to 9, the symmetrical regular distribution of the perforations guarantees that several of these positions exist. The means defining, between the said first 1 and second 2 parts, a cavity sealed against the surrounding materials are for example constituted by an annular recess 431 made in the said first part 1 on the face in contact with the said second part 2, centred on the axis of rotation of the two said first 1 and second 2 parts, and which receives for example an O-ring joint made of silicon elastomer 432. The operation of this third embodiment can be easily deduced from that of the previous ones. FIGS. 8 and 9 represent respectively the device in its free and blocked states.

The fourth embodiment of the device according to the invention represented in FIGS. 10 to 14 illustrates an example of means to hold the said blocking part in at least one of the said first free state and second blocked state.

These means can be of any kind and notably:
elastic means, such as a bistable blade which holds in one or other of the free or blocked states the said blocking part until a sufficient force has been exerted. These means will allow, for example, a transition from one state to the other under the effect of gravity to be prevented, and to be permitted only under the effect of a sufficient magnetic field.
magnetic: small magnets placed in the said first or the said second part hold in the corresponding state the said blocking part which in this case comprises an element on which a magnetic field is able to exert a force. The advantage and operation are similar to those of the elastic means described above.
adhesive: an adhesive for single use or multiple use, according to the desired operation, is placed judiciously on one of the said first or second parts.
geometrical,

The said fourth embodiment illustrates the means of geometrical nature which are of particular interest. Indeed, the device according to the invention in this fourth embodiment can change from the free state to the blocked state only if the orientation of the said device in the field which applies a force to the blocking part is made starting from a particular position and according to a stipulated trajectory. Thus, for example, if the said fourth embodiment of the device is used and suitably arranged in a femur prosthesis, the wearer of the prosthesis can make all the movements of day-to-day life without any risk of involuntary lengthening and only a movement such as that obtained by changing directly from the standing position or lying on the stomach to that of “walking on their hands”, which is very specific and generally requires the assistance of a third party, allows the said first 1 and second 2 parts to be blocked, and lengthening to be caused by activating a mechanism which transforms, for example, the alternative rotations of the second part 2 into a lengthening.

Indeed, as for the third embodiment, the said first 1 and second 2 parts are in contact through faces perpendicular to their common axis of rotation. Blocking part 3 is a rectangular parallelepiped. The second blocking surfaces 21 are constituted by faces of a parallelepiped cavity made in the second part 2 and which can half contain, with a small amount of play, the said blocking part. The first blocking surfaces 11 are constituted by the faces of a perforation of rectangular section which can in an angular position at least of the said first 1 and second 2 parts face the said second blocking surfaces 21. But, in this fourth embodiment, an additional parallelepiped cavity 52, widens at the bottom and on one side the cavity limited by the said first blocking surfaces 11 over a sufficient width, length and depth to fully or almost contain the said blocking part 3.

If the said cavity 52 is overlooked, the fourth embodiment of the said device according to the invention operates like the previous embodiments.

Conversely, if one observes the properties of the said fourth embodiment if its initial position is the free state, and such that the blocking part 3 is located in the said cavity 52 as in FIG. 11, causing the first part 1 to pass above the second part 2 does not cause the transition to the blocked state since the said blocking part 3 remains blocked on edge 51 of the said cavity 52 as in FIG. 12. To cause this transition to the blocked state from the said position of FIG. 11, it is necessary firstly to rotate the said fourth embodiment by 180 degrees around the axis of rotation of the said first 1 and second 2 parts, or indeed at least the said first part 1 alone by this angle, if this is possible, to release the said blocking part 3 from the said cavity 52 as in FIG. 13, and then to do as when the said cavity 52 is absent to obtain the blocked state of FIG. 14. Operation in the opposite direction, from the blocked state to the free state, is normal.

The means defining between the said first 1 and second 2 parts a cavity sealed against the surrounding materials are not represented for this fourth embodiment. They can be of any type.

The device according to the invention has the advantage that it may be made from all types of materials, particularly all materials known to be implantable such as certain cobalt-and chromium-based alloys or high-performance polymers such as polyetheretherketone, for example, and that it may be incorporated in any environment if it operates by gravity. It is however necessary that the system in which it is incorporated is constituted essentially of materials which are insensitive to magnetic and non-magnetic fields if it comprises such itself, in order to be able to control it with such fields.

There are many applications of the device according to the invention: it is incorporated in existing systems in order to improve their control, and also allows new systems to be conceived.

In an existing system in which the alternative rotation of a shaft modifies the said system as is the case for several bone-lengthening nails, for example, the device according to the invention enables the said shaft to be converted into a shaft in two parts, which then constitute the first and second parts as defined in the invention, separated by a clutch with a non-invasive control constituted by the said device according to the invention, or also allows blocking and unblocking of rotation of the said shaft, which remains in a single part, wherein the first part is as defined in the invention, relative to the reference part, and the second part is according to the case and the user's choice.

The device according to the invention will thus advantageously be used to control satisfactorily or even suspend the lengthening obtained by a rotary nail, or an expandable prosthesis, or to block a prosthetic joint of the knee in a position, generally for extension, in the event that the soft tissues of the patient do not allow, temporarily or definitively, normal activation of the knee and its stability.

Beyond modifications of systems of known types, other new systems will easily be able to be conceived through the use of the new possibilities offered by the said device according to the invention and, for example, prostheses allowing non-invasive modification after their implantation of angles such as external rotation or the valgus of the receiving limb, and other new implantable systems with adjustable geometry.

More generally, the device according to the invention may be used to control by simple orientation in space or in a magnetic field every system driven by an alternative rotational movement. It will thus be possible to produce games and toys (for example an automaton which simulates walking with legs if it has the feet “down” and walking on the hands in the contrary case, or thinking and manipulation games) or again security systems (for example, a screw-based sealing system which can be unscrewed only if it is oriented suitably relative to gravity) using the device according to the invention.

1. A device according to the invention to block and unblock in a non-invasive manner the rotation between two first (1) and second (2) parts implanted in the organism wherein:
first blocking surfaces (11) bound to the said first part (1),
second blocking surfaces (21) bound to the said second part (2),
at least one blocking part (3):
which comprises third blocking surfaces (31), able to cooperate with the said first blocking surfaces (11) which are bound to the said first part (1) and the fourth blocking surfaces (32), able to cooperate with the said second blocking surfaces (21) bound to the said second part (2), and
capable, when both the said first (1) and second (2) parts are at a particular angular position, of passing from a first free state in which both the said first (1) and second (2) parts are free to rotate relative to one another, with at least one of the said third (31) and fourth (32) blocking surfaces of the said blocking part being free from the blocking surfaces (11, 21) of that of the said first (1) and second (2) parts with which they can cooperate, to a second blocked state in which the said first (1) and second (2) parts are prevented from rotating relative to one another by means of the said first (11) and third (31) blocking surfaces firstly, and of the said second (21) and fourth (32) blocking surfaces secondly, and vice-versa to pass from the said second blocked state to the said first free state;
means (411, 412, 413, 421, 422, 431, 432) defining between the said first (1) and second (2) parts a cavity sealed against the surrounding materials which includes at least the said first (11) and second (21) blocking surfaces and the volume swept by the said blocking part (3) when it moves between the said first free state and the said second blocked state, and vice-versa, and
means to impart rotation in one direction and in the other from outside the organism to the said first and second parts relative to one another.

2. A device according to claim 1, wherein at least one blocking part (3) comprises at least one element on which a magnetic field is able to exert a force.

3. A device according to claim 1, wherein the said means defining between the first and second parts a cavity sealed against the surrounding materials comprise at least one flexible conduit (411) bound at one of its ends to the said first part (1) and at the other of its ends to the said second part (2).

4. A device according to claim 1, wherein it also comprises means (51, 52) to hold at least one blocking part (3) in at least one of the said first free state and second blocked state.

5. A device according to claim 1, wherein the means to impart rotation in one direction and in the other from outside the organism to the two said first (1) and second (2) parts comprise means to link to at least one bone segment.

6. A device according to claim 1, wherein the means to impart rotation in one direction and in the other from outside the organism to the two said first and second parts comprise means placed in the soft tissues.

7. A device according to claim 1, wherein the means to impart rotation in one direction and in the other from outside the organism to the two said first and second parts comprise elastic means.
